(19) United States
(12) Patent Application Publication
(10) Pub. No.: US 2021/0275612 A1
(43) Pub. Date: Sep. 9, 2021

(54) METHOD FOR ALLEVIATING INFLAMMATION-RELATED DISORDER

(71) Applicant: GLAC BIOTECH CO., LTD., Tainan City (TW)

(72) Inventors: Cheng-Ruei LIU, Tainan City (TW); Yi-Wei KUO, Tainan City (TW); Hsieh-Hsun HO, Tainan City (TW)

(21) Appl. No.: 17/191,766

(22) Filed: Mar. 4, 2021

(30) Foreign Application Priority Data
Mar. 9, 2020 (TW) 109107660

(51) Int. Cl. A61K 35/747; A61K 35/745; A61P 37/06; A23L 33/135; A23L 33/00

(52) U.S. Cl. CPC A61K 35/747; A61K 35/745; A61P 37/06; A23L 33/135; A23V 2002/00; A23Y 2220/67; A23Y 2300/49; A23Y 2300/55; A23L 33/40

(57) ABSTRACT

Disclosed herein is a method for alleviating an inflammation-related disorder, which includes administering to a subject in need thereof a composition containing a culture of at least one lactic acid bacterial strain. The at least one lactic acid bacterial strain is selected from the group consisting of Lactobacillus plantarum LPL28 which is deposited at the China General Microbiological Culture Collection Center (CGMCC) under an accession number CGMCC 17954, Bifidobacterium animalis subsp. lactis CP-9 which is deposited at the China Center for Type Culture Collection (CCTCC) under an accession number CCTCC M 2014588, and Bifidobacterium longum subsp. longum OLP-01 which is deposited at the CGMCC under an accession number CGMCC 17345.

### CROSS-REFERENCE TO RELATED APPLICATION

This application claims priority of Taiwanese Invention Patent Application No. 109107660, filed on Mar. 9, 2020.

### FIELD

The present disclosure relates to a method for alleviating an inflammation-related disorder using a composition containing a culture of lactic acid bacteria.

### BACKGROUND

Probiotics are resident normal flora of the intestinal tract and believed to play important roles in regulating proper intestinal immunity and digestion by balancing intestinal microflora. These beneficial microorganisms are widely used as live microbial dietary supplements and can help restoring intestinal microfloral balance. Many species of lactic acid bacteria (LAB) are conferred with the generally recognized as safe (GRAS) status, and are widely used as probiotics.

Common LAB include Lactobacillus spp., Lactococcus spp., Pediococcus spp., Streptococcus spp., Enterococcus spp., Bifidobacterium spp., Bacillus spp., Leuconostoc spp., etc. LAB have been shown to be capable of inhibiting the growth of pathogenic bacteria in the gastrointestinal tract and alleviating lactose intolerance, and to have anti-cancer, anti-bacterial, anti-fatigue, and blood pressure lowering effects.

It has been reported in Lee M. C. et al. (2019), Nutrients, doi: 10.3390/nu11092003 that Bifidobacterium longum subsp. longum OLP-01 supplementation can significantly decrease lactate, ammonia, blood urea nitrogen (BUN), and creatine kinase (CK) levels. In addition, Bifidobacterium longum subsp. longum OLP-01 supplementation can also increase the resting levels of both hepatic and muscular glycogen.

The results indicate that Bifidobacterium longum subsp. longum OLP-01 supplementation is effective in promoting health, improving exercise performance, and ameliorating fatigue.

In the applicant's previous study, a respective one of Lactobacillus plantarum LPL28 and Bifidobacterium animalis subsp. lactis CP-9 was fermented with lactose and a whey protein concentrate or a complex culture medium, and the corresponding resultant LAB whey isolate (LWI) and LAB fermentation broth (LFB) were subjected to antimicrobial activity analysis. The results show that Lactobacillus plantarum LPL28 and Bifidobacterium animalis subsp lactis CP-9 have obvious bacteriostatic activity on all vaginal pathogens (Hsieh P. S. et al. (2017), “Comparison of the Inhibition Effect of Lactic Acid Bacteria Fermentation Metabolites on Different Vaginal Pathogens and Developing Gynecological Probiotic Products,” International Proceedings of Conference Full-text Database (IPFD)).

Inflammation-related disorders are the most significant cause of death in the world.

Inflammation-related disorders have a broad spectrum involvement in many human acute and chronic diseases including allergies, arthritis and joint diseases, diabetes, cardiovascular diseases, chronic obstructive pulmonary disease (COPD), and autoimmune disorders. Therapeutic management of inflammation-related disorders has focused on the development of anti-inflammatory drugs that block cytokine signaling. Currently, drugs used clinically to alleviate inflammation-related disorders include immunosuppressive drugs (such as cyclosporin) and anti-inflammatory drugs (such as corticosteroids). However, these drugs might not be able to achieve the desired therapeutic effect and may also cause severe side effects. Therefore, there is still a need to develop a new strategy that can be utilized in the alleviation of inflammation-related disorders.

### SUMMARY

Accordingly, an object of the present disclosure is to provide a method for alleviating an inflammation-related disorder that can alleviate at least one of the drawbacks of the prior art.

The method includes administering to a subject in need thereof a composition containing a culture of at least one lactic acid bacterial strain,

wherein the at least one lactic acid bacterial strain is selected from the group consisting of Lactobacillus plantarum LPL28 which is deposited at the China General Microbiological Culture Collection Center (CGMCC) under an accession number CGMCC 17954, Bifidobacterium animalis subsp. lactis CP-9 which is deposited at the China Center for Type Culture Collection (CCTCC) under an accession number CCTCC M 2014588, and Bifidobacterium longum subsp. longum OLP-01 which is deposited at the CGMCC under an accession number CGMCC 17345.

### BRIEF DESCRIPTION OF THE DRAWINGS

Other features and advantages of the present disclosure will become apparent in the following detailed description of the embodiments with reference to the accompanying drawings, of which:

FIG. 1 shows the interleukin-10 (IL-10) content in each group of Example 2, infra, in which the symbol “***” represents p<0.001 (compared with the comparative group); and

FIG. 2 shows the transforming growth factor-β (TGF-β) contents in the control group and experimental groups 1 to 5 of Example 2, infra, in which the symbol “***” represents p<0.001 (compared with the control group).

### DETAILED DESCRIPTION

It is to be understood that, if any prior art publication is referred to herein, such reference does not constitute an admission that the publication forms a part of the common general knowledge in the art, in Taiwan or any other country.

For the purpose of this specification, it will be clearly understood that the word “comprising” means “including but not limited to”, and that the word “comprises” has a corresponding meaning.

Unless otherwise defined, all technical and scientific terms used herein have the meaning commonly understood by a person skilled in the art to which this disclosure belongs. One skilled in the art will recognize many methods and materials similar or equivalent to those described herein, which could be used in the practice of this disclosure. Indeed, this disclosure is in no way limited to the methods and materials described.

The present disclosure provides a method for alleviating an inflammation-related disorder, which includes administering to a subject in need thereof a composition containing a culture of at least one lactic acid bacterial strain,

wherein the at least one lactic acid bacterial strain is selected from the group consisting of Lactobacillus plantarum LPL28 which is deposited at the China General Microbiological Culture Collection Center (CGMCC) under an accession number CGMCC 17954, Bifidobacterium animalis subsp. lactis CP-9 which is deposited at the China Center for Type Culture Collection (CCTCC) under an accession number CCTCC M 2014588, and Bifidobacterium longum subsp. longum OLP-01 which is deposited at the CGMCC under an accession number CGMCC 17345.

As used herein, the term “alleviating” or “alleviation” refers to at least partially reducing, ameliorating, relieving, controlling, treating or eliminating one or more clinical signs of a disease or disorder; and lowering, delaying, stopping or reversing the progression of severity regarding the condition or symptom being treated and preventing or decreasing the likelihood or probability thereof.

As used herein, the term “administering” or “administration” means introducing, providing or delivering the abovementioned composition to a subject showing condition(s) or symptom(s) of an inflammation-related disorder by any suitable routes to perform its intended function.

As used herein, the term “subject” refers to any animal of interest, such as humans, monkeys, cows, sheep, horses, pigs, goats, dogs, cats, mice, and rats. In certain embodiments, the subject is a human.

According to the present disclosure, the inflammation-related disorder may be selected from the group consisting of allergy, organ transplant rejection, systemic lupus erythematosus, rheumatoid arthritis, psoriasis, multiple sclerosis, inflammatory bowel disease, Graves' disease, celiac disease, type 1 diabetes mellitus, and combinations thereof.

According to the present disclosure, the culture of the lactic acid bacterial strain is prepared by culturing the abovementioned at least one lactic acid bacterial strain in a liquid or solid medium suitable for growth and/or proliferation thereof.

As used herein, the term “culturing” can be used interchangeably with other terms such as “fermentation” and “cultivation”.

According to the present disclosure, the liquid medium suitable for culturing the lactic acid bacterial strain may include, but is not limited to, MRS (De Man, Rogosa and Sharpe) broth and a medium containing milk whey (MMW).

According to the present disclosure, the liquid medium suitable for culturing the lactic acid bacterial strain may include a carbon source selected from the group consisting of glucose, fructose, lactose, sucrose, maltose, galactose, mannose, trehalose, starch, molasses, potato starch, corn starch, malt extract, maltodextrin, and combinations thereof.

In certain embodiments, the liquid medium may include, based on the total weight of the liquid medium, 2% to 5% of a carbon source mixture of glucose and maltodextrin. In an exemplary embodiment, the liquid medium includes, based on the total weight of the liquid medium, 3% of a carbon source mixture of glucose and maltodextrin.

According to the present disclosure, the liquid medium suitable for culturing the lactic acid bacterial strain may include a nitrogen source selected from the group consisting of ammonium sulfate ((NH₄)₂SO₄), ammonium phosphate ((NH₄)₃PO₄), ammonium nitrate (NH₄NO₃), ammonium chloride (NH₄Cl), casamino acid, urea, peptone, polypeptone, tryptone, meat extract, yeast extract, yeast powders, cow's milk, milk powders, soy products (e.g., soybean flour), whey, and combinations thereof.

In certain embodiments, the liquid medium may include, based on the total weight of the liquid medium, 5% to 30% of cow's milk and 1% to 10% of soybean flour.

According to the present disclosure, the solid medium suitable for culturing the lactic acid bacterial strain may include the following components: peptone, yeast extract, beef extract, dextrose, sodium acetate, polysorbate 80, K₂HPO₄, MnSO₄.4H₂O, MgSO₄.7H₂O, ammonium citrate, agar, and water.

The procedures and conditions for culturing the lactic acid bacterial strain may be adjusted according to practical requirements. In this regard, those skilled in the art may refer to journal articles, e.g., Hsieh P. S. et al. (2013), New Microbiol., 36:167-179.

In certain embodiments, the lactic acid bacterial strain may be cultured at a temperature ranging from 25° C. to 40° C. In an exemplary embodiment, the lactic acid bacterial strain is cultured at 37° C.

In certain embodiments, the lactic acid bacterial strain may be cultured for a time period ranging from 20 hours to 40 hours. In an exemplary embodiment, the lactic acid bacterial strain is cultured for 24 hours.

In certain embodiments, the culture of the lactic acid bacterial strain is a liquid culture.

In certain embodiments, the liquid culture may have a total bacterial concentration not greater than 109 CFU/mL. In an exemplary embodiment, the liquid culture is substantially free of cells.

As used herein, the term “substantially free of” means that the liquid culture lacks a significant amount of a specified component (i.e., lactic acid bacterial cells). In certain embodiments, the amount of the lactic acid bacterial cells does not have a measurable effect on the properties of the liquid culture. In other embodiments, the liquid culture is completely free of the bacterial cells.

According to the present disclosure, the liquid culture which is substantially free of cells is obtained by subjecting a culture formed after culturing the lactic acid bacterial strain to a separation treatment to remove bacterial cells therefrom.

According to the present disclosure, the separation treatment may be performed using techniques well-known to those skilled in the art. Examples of the separation treatment may include, but are not limited to, filtration, centrifugation, concentration, and combinations thereof.

In an exemplary embodiment, the liquid culture which is substantially free of cells is obtained by subjecting the culture formed after culturing the lactic acid bacterial strain to a centrifugation treatment.

According to the present disclosure, after the separation process, a drying process may be performed using techniques well-known to those skilled in the art, so as to obtain a product in a powder form. Examples of the drying process may include, but are not limited to, a lyophilization treatment, a low temperature spray-drying treatment, a vacuum evaporation treatment, and combinations thereof.

According to the present disclosure, the composition may be formulated as a food product using a standard technique well known to one of ordinary skill in the art. For example, the composition may be directly added to an edible material or may be used to prepare an intermediate composition (e.g., a premix) suitable to be subsequently added to the edible material.

As used herein, the term “food product” refers to any article or substance that can be ingested by a subject into the body thereof. Examples of the food product may include, but are not limited to, milk powders, fermented milk, yogurt, butter, beverages (e.g., tea, coffee, etc.), functional beverages, a flour product, baked foods, confectionery, candies, fermented foods, animal feeds, health foods, and dietary supplements.

According to the present disclosure, the food product may further include an additional food additive selected from the group consisting of starch, dextrin, lactose, maize flour, rice flour, tricalcium phosphate, silicon dioxide, magnesium stearate, calcium carbonate, glucose, sucrose, fructose, sugar alcohol, oligosaccharides, sugar substitutes, fruit juice powders, yeast powders, nonfat dry milk, casein, whey proteins, amino acids, citric acid, citrate, lactic acid, lactate, nucleotides, and combinations thereof.

According to the present disclosure, the composition may be prepared in the form of a pharmaceutical composition.

According to the present disclosure, the pharmaceutical composition may be formulated into a suitable dosage form for oral or topical administration using technology well known to those skilled in the art.

According to the present disclosure, the suitable dosage form for oral administration includes, but is not limited to, sterile powders, tablets, troches, lozenges, pellets, capsules, dispersible powders or granules, solutions, suspensions, emulsions, syrup, elixir, slurry, drops, and the like.

According to the present disclosure, the suitable dosage form for topical administration includes, but is not limited to, emulsions, gels, ointments, creams, patches, liniments, powders, aerosols, sprays, lotions, serums, pastes, foams, drops, suspensions, salves, and bandages.

According to the present disclosure, the pharmaceutical composition may further include a pharmaceutically acceptable carrier widely employed in the art of drug-manufacturing. For instance, the pharmaceutically acceptable carrier may include one or more of the following agents: solvents, buffers, emulsifiers, suspending agents, decomposers, disintegrating agents, dispersing agents, binding agents, excipients, stabilizing agents, chelating agents, diluents, gelling agents, preservatives, wetting agents, lubricants, absorption delaying agents, liposomes, and the like. The choice and amount of the aforesaid agents are within the expertise and routine skills of those skilled in the art.

According to the present disclosure, the pharmaceutical composition may further include a probiotic microorganism selected from the group consisting of Lactobacillus acidophilus TYCA06 (BCRC 910813), Lactobacillus reuteri GL-104 (BCRC 910404), Lactobacillus salivarius subsp. Salicinius AP-32 (BCRC 910437), Lactobacillus rhamnosus F-1 (BCRC 910469), Bifidobacterium longum subsp. infantis BLI-02 (BCRC 910812), and combinations thereof.

The dose and frequency of administration of the composition may vary depending on the following factors: the severity of the illness or disorder to be treated, routes of administration, and age, physical condition and response of the subject to be treated. In general, the composition may be administered in a single dose or in several doses.

The present disclosure will be further described by way of the following examples. However, it should be understood that the following examples are intended solely for the purpose of illustration and should not be construed as limiting the present disclosure in practice.

### EXAMPLES

### General Experimental Materials:

### 1. Preparation of Human Peripheral Blood Mononuclear Cells (Human PBMCs)

Blood samples were collected from healthy human volunteers aged between 20 and 40 years (employees of Glac Biotech Co., Ltd., Taiwan), and an acid citrate dextrose (ACD) solution was used as an anticoagulant. The blood samples were subjected to density gradient centrifugation (720 g, 30 minutes) at 4° C. with Ficoll-Paque” PLUS. Thereafter, the lymphocyte layer was harvested, followed by adding a red blood cell (RBC) lysis buffer. After centrifugation at 3,000 rpm and 4° C. for 10 minutes, the red blood cells were removed, so as to obtain human PBMCs, and the cell concentration was adjusted to 4×10⁶ cells/mL using an RPMI 1640 medium containing 10% fetal bovine serum (FBS).

2. Bifidobacterium bifidum gL-306 and Bifidobacterium breve gL-57 Used in the Following Experiments were Isolated from Human Breast Milk by the Applicant.

### 3. Lactic Acid Bacterial (LAB) Strains

(1)Lactobacillus plantarum LPL28

Lactobacillus plantarum LPL28 has been deposited at the China General Microbiological Culture Collection Center (CGMCC) under an accession number CGMCC 17954 since Jun. 18, 2019.

(2)Bifidobacterium animalis subsp. lactis CP-9

Bifidobacterium animalis subsp. lactis CP-9, which is disclosed in the applicant's previous Taiwanese Invention Patent Publication No. 1572713, has been deposited at the BCRC (Biosource Collection and Research Center) of the FIRDI (Food Industry Research and Development Institute) in Taiwan under accession number BCRC 910645 since Aug. 21, 2014, and has also been deposited at the China Center for Type Culture Collection (CCTCC) under an accession number CCTCC M 2014588 since Nov. 24, 2014.

(3)Bifidobacterium longum subsp. longum OLP-01

Bifidobacterium longum subsp. longum OLP-01 has been deposited at the CGMCC under an accession number CGMCC 17345 since Mar. 18, 2019.

### General Procedures:

### 1. Statistical Analysis

All the experiments described below were performed in triplicate. The experimental data are expressed as mean±standard error of the mean (SEM) and were analyzed using two-tailed Student's t-test so as to assess the difference between all the groups. Statistical significance is indicated by p<0.05.

### Example 1. Preparation of Liquid Culture of Lactic Acid Bacterial (LAB) Strain of the Present Disclosure

A respective one of Bifidobacterium bifidum gL-306, Bifidobacterium breve gL-57, Lactobacillus plantarum LPL28, Bifidobacterium animalis subsp. lactis CP-9, and Bifidobacterium longum subsp. longum OLP-01 was inoculated in a MRS broth (BD Biosciences), and was then cultivated in an incubator (37° C., 5% CO₂) for 20 to 24 hours to obtain a respective inoculum. Thereafter, the respective inoculum was inoculated in an amount of 1 to 3% (v/v) into a MRS medium (supplemented with 5% to 30% of cow's milk, 1% to 10% of soybean flour, and 3% of a carbon source mixture of glucose and maltodextrin), and was then cultured at 37° C. for 24 hours.

After centrifugation at 3,000 rpm to 6,000 rpm for 30 minutes, the resultant cell culture supernatant was collected and then subjected to sterilization by heating, followed by a spray-drying treatment, so as to obtain a respective one of the following: a spray-dried powder of Bifidobacterium bifidum gL-306 cell culture supernatant, a spray-dried powder of Bifidobacterium breve gL-57 cell culture supernatant, a spray-dried powder of Lactobacillus plantarum LPL28 cell culture supernatant, a spray-dried powder of Bifidobacterium animalis subsp. lactis CP-9 cell culture supernatant, and a spray-dried powder of Bifidobacterium longum subsp. longum OLP-01 cell culture supernatant.

A respective one of the five spray-dried powders was mixed with a suitable amount of an RPMI 1640 medium to reach a final concentration of 30 g/L, and the resultant liquid culture test samples were subjected to the following experiments.

### Example 2. Evaluation for the Effect of Liquid Culture of LAB Strain According to this Disclosure on Alleviating Inflammation

### A. Treatment of Human PBMCs Using Liquid Culture of LAB Strain

The human PBMCs prepared in section 1 of “General Experimental Materials” were divided into 7 groups, including one control group, one comparative group, and five experimental groups (i.e., experimental groups 1 to 5). Each group of the human PBMCs was incubated in a respective well of a 96-well culture plate containing 200 μL of an RPMI 1640 medium (supplemented with 10% FBS and 1% penicillin-streptomycin (PS)) at 1×10⁵ cells/well, followed by cultivation in an incubator (37° C., 5% CO₂) for 48 hours.

Afterwards, the culture medium in each well was removed. The cells of each of the experimental groups 1 to 5 were added with a suitable amount of an RPMI 1640 medium containing 30 g/L of a respective one of the five liquid culture test samples prepared in Example 1. In addition, the cells of the comparative group were added with a suitable amount of an RPMI 1640 medium containing 200 ng/mL of phytohemagglutinin (PHA), and the cells of the control group were added with a suitable amount of a regular RPMI 1640 medium only.

The treating component and the final concentration thereof for each group are summarized in Table 1 below.

TABLE 1

| Group | Treating component | Final concentration | |
|---|---|---|---|
| Control group | — | 0 | |
| Comparative group | PHA | 200 | ng/mL |
| Experimental group 1 | Liquid culture test | 30 | g/L |
| | sample of Lactobacillus | | |
| | plantarum LPL28 | | |
| Experimental group 2 | Liquid culture test | 30 | g/L |
| | sample of Bifidobacterium | | |
| | animalis subsp. lactis | | |
| | CP-9 | | |
| Experimental group 3 | Liquid culture test | 30 | g/L |
| | sample of Bifidobacterium | | |
| | longum subsp. longum | | |
| | OLP-01 | | |
| Experimental group 4 | Liquid culture test | 30 | g/L |
| | sample of Bifidobacterium | | |
| | bifidum gL-306 | | |
| Experimental group 5 | Liquid culture test | 30 | g/L |
| | sample of Bifidobacterium | | |
| | breve gL-57 | | |

Each group was cultivated in an incubator (37° C., 5% CO₂) for 48 hours. After centrifugation at 3,000 rpm for 10 minutes, the resultant supernatant was collected, and was subjected to the following experiments.

### B. Effect of Liquid Culture of LAB Strain on Interleukin-10 (IL-10) Content of Human PBMCs

The supernatant of each group prepared in section A of this example was subjected to determination of IL-10 content. The IL-10 content was determined using an enzyme-linked immunosorbent assay (ELISA) kit (Cat. No. 88-7106-88, eBioscience Inc.) in accordance with the manufacturer's instructions.

The data thus obtained were analyzed according to the method described in section 1 of “General Procedures”.

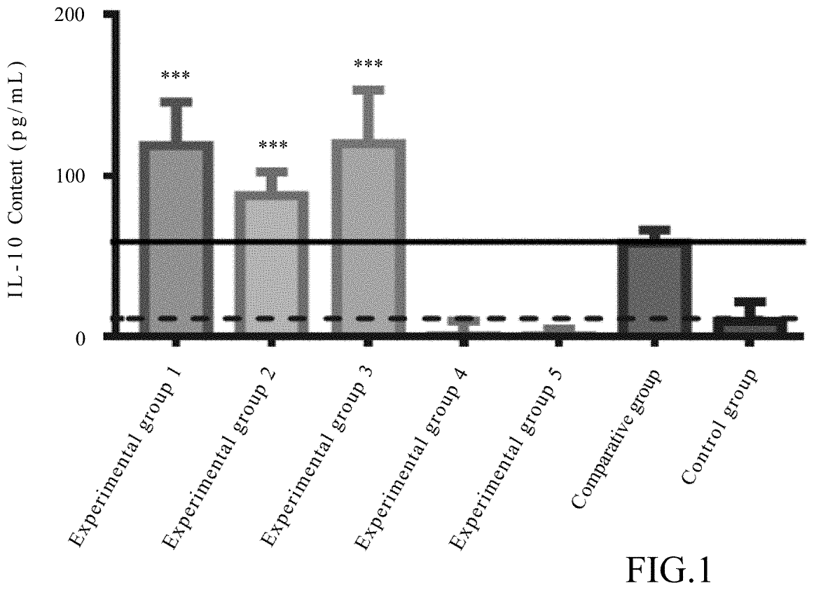

Referring to FIG. 1, the IL-10 contents determined in the experimental groups 1 to 3 were each higher than those determined in the experimental groups 4 to 5, the comparative group, and the control group. This result suggests that the liquid culture of any one of Lactobacillus plantarum LPL28, Bifidobacterium animalis subsp. lactis CP-9, and Bifidobacterium longum subsp. longum OLP-01 is effective in inducing IL-10 secretion in human PBMCs.

### C. Effect of Liquid Culture of LAB Strain on Transforming Growth Factor-β (TGF-β) Content of Human PBMCs

Each of the supernatants of the control group and five experimental groups prepared in section A of this example was subjected to determination of TGF-β content. The TGF-β content was determined using an ELISA kit (Cat. No. 88-8350-88, eBioscience Inc.) in accordance with the manufacturer's instructions.

The data thus obtained were analyzed according to the method described in section 1 of “General Procedures”.

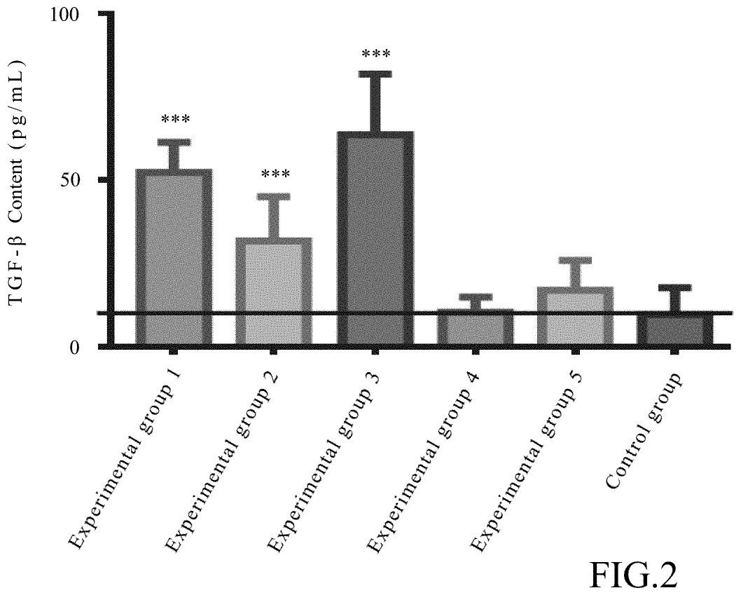

Referring to FIG. 2, the TGF-0 contents determined in the experimental groups 1 to 3 were each higher than those determined in the experimental groups 4 to 5 and the control group. This result suggests that the liquid culture of any one of Lactobacillus plantarum LPL28, Bifidobacterium animalis subsp. lactis CP-9, and Bifidobacterium longum subsp. longum OLP-01 is effective in inducing TGF-β secretion in human PBMCs.

Summarizing the above test results, it is clear that the abovementioned liquid culture of at least one lactic acid bacterial strain of this disclosure (i.e. Lactobacillus plantarum LPL28, Bifidobacterium animalis subsp. lactis CP-9, and Bifidobacterium longum subsp. longum OLP-01) can increase the anti-inflammation-associated IL-10 and TGF-0, and hence can alleviate an inflammation-related disorder.

In the description above, for the purposes of explanation, numerous specific details have been set forth in order to provide a thorough understanding of the embodiments. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A method for alleviating an inflammation-related disorder, comprising administering to a subject in need thereof a composition containing a culture of at least one lactic acid bacterial strain,
wherein the at least one lactic acid bacterial strain is selected from the group consisting of Lactobacillus plantarum LPL28 which is deposited at the China General Microbiological Culture Collection Center (CGMCC) under an accession number CGMCC 17954, Bifidobacterium animalis subsp. lactis CP-9 which is deposited at the China Center for Type Culture Collection (CCTCC) under an accession number CCTCC M 2014588, and Bifidobacterium longum subsp. longum OLP-01 which is deposited at the CGMCC under an accession number CGMCC 17345.

2. The method as claimed in claim 1, wherein the inflammation-related disorder is selected from the group consisting of allergy, organ transplant rejection, systemic lupus erythematosus, rheumatoid arthritis, psoriasis, multiple sclerosis, inflammatory bowel disease, Graves' disease, celiac disease, type 1 diabetes mellitus, and combinations thereof.

3. The method as claimed in claim 1, wherein the culture is a liquid culture.

4. The method as claimed in claim 3, wherein the liquid culture is substantially free of cells.

5. The method as claimed in claim 1, wherein the composition is formulated as a food product.

6. The method as claimed in claim 1, wherein the composition is formulated as a pharmaceutical composition.

7. The method as claimed in claim 6, wherein the pharmaceutical composition is in a dosage form for oral administration.

8. The method as claimed in claim 6, wherein the pharmaceutical composition is in a dosage form for topical administration.

9. The method as claimed in claim 1, wherein the composition further comprises a probiotic microorganism selected from the group consisting of Lactobacillus acidophilus TYCA06 (BCRC 910813), Lactobacillus reuteri GL-104 (BCRC 910404), Lactobacillus salivarius subsp. salicinius AP-32 (BCRC 910437), Lactobacillus rhamnosus F-1 (BCRC 910469), Bifidobacterium longum subsp. infantis BLI-02 (BCRC 910812), and combinations thereof.
